(19) United States
(12) Patent Application Publication
(10) Pub. No.: US 2016/0346072 A1
(43) Pub. Date: Dec. 1, 2016

(54) PROTECTION BODY AND MEDICAL DEVICE

(71) Applicant: TERUMO KABUSHIKI KAISHA, Tokyo (JP)

(72) Inventor: Yoshio KAWASHIMA, Fujinomiya (JP)

(73) Assignee: TERUMO KABUSHIKI KAISHA, Tokyo (JP)

(21) Appl. No.: 15/231,814

(22) Filed: Aug. 9, 2016

Related U.S. Application Data
(63) Continuation of application No. PCT/JP2015/050389, filed on Jan. 8, 2015.

(30) Foreign Application Priority Data
Feb. 17, 2014 (JP) 2014-027567

(51) Int. Cl. A61F 2/00; A61B 50/30; A61F 2/95

(52) U.S. Cl. CPC A61F 2/0095; A61F 2/95; A61B 50/30; A61F 2250/0067; A61B 2050/3004

(57) ABSTRACT

A protection body protecting the outer circumference of a drug retaining portion, which is provided at a distal portion of a medical elongated body and in which a drug is retained, by covering and sealing the outer circumference, includes a protection layer which is disposed around the outer circumference of the drug retaining portion along the medical elongated body and prevents external oxygen and moisture from coming into contact with the drug retaining portion; and an absorption portion which absorbs oxygen and moisture in a space in the inner circumference of the protection layer and in the outer circumference of the drug retaining portion.

### CROSS-REFERENCES TO RELATED APPLICATIONS

This application is a continuation of International Application No. PCT/JP2015/050389 filed on Jan. 8, 2015, and claims priority to Japanese Patent Application No. 2014-027567 filed on Feb. 17, 2014, the entire content of each of which is incorporated herein by reference.

### TECHNICAL FIELD

The present invention relates to a protection body and a medical device.

### BACKGROUND DISCUSSION

In order to reduce the burden on a patient during a surgical operation, it is known to use a medical tool such as a catheter. For example, in percutaneous transluminal coronary angioplasty (PTCA) used in cardiac infarction or angina, a stent is transported to a stenosed site within a blood vessel using a balloon catheter, and a balloon is dilated at a point in time when the stent reaches the stenosed site. Occurrence of restenosis in a lesion area is suppressed by indwelling the stent, which has been expanded by the dilation of the balloon, in the stenosed site.

In addition, a drug eluting stent (DES), which prevents restenosis by making the outer surface of this stent carry a biologically/physiologically active substance such as an anticancer drug (hereinafter, referred to as a “drug”), and eluting the drug after the indwelling of the stent, has been developed. However, such a drug easily reacts with oxygen or moisture and causes an oxidation reaction or a hydrolysis reaction. Therefore, it is desirable to store the drug so as not to come into contact with oxygen or moisture until the drug is used during operation.

In this context, a method for, for example, storing the entirety of a catheter including a drug eluting stent in an aluminum bag in which a deoxidant and a desiccant are included is disclosed in Japanese Patent No. 5008555. In addition, a method for purging the inside of the aluminum bag with nitrogen in order to remove oxygen and moisture in the aluminum bag is known. According to this storage method, since the bag is made of aluminum, it is possible to prevent oxygen and moisture from entering the bag from the outside. In addition, a deoxidant and a desiccant are stored in the aluminum bag while the aluminum bag is purged with nitrogen, and therefore, it is possible to maintain the inside of the bag to be in low-oxygen and low-humidity states. Accordingly, it is possible to reduce a possibility that the drug may react with oxygen or moisture and cause an oxidation reaction or a hydrolysis reaction.

### SUMMARY

However, the above-described storage method uses a manufacturing method in which a deoxidant and a desiccant are included in the aluminum bag which also stores a medical device including a catheter, and in which the entirety of the inside of the aluminum bag is purged with nitrogen. Such a manufacturing method can be complicated and costly. Disclosed herein is a protection body and a medical device which can easily prevent a drug for preventing restenosis of a lesion area from reacting with oxygen and moisture and which can be manufactured relatively easily and inexpensively.

A protection body according to the present disclosure can protect the outer circumference of a drug retaining portion which is provided at a distal portion of a medical elongated body and in which a drug is retained, by covering and sealing the outer circumference. The protection body has a protection layer which is disposed around the outer circumference of the drug retaining portion along the medical elongated body and can prevent external oxygen and moisture from coming into contact with the drug retaining portion, and an absorption portion which can absorb oxygen and moisture in a space between the inner circumference of the protection layer and the outer circumference of the drug retaining portion.

In addition, a medical device according to the present disclosure includes the above-described protection body, and a medical elongated body including a drug retaining portion, in which a drug is retained, at a distal portion, in which the inner circumference of the protection body on a distal side is liquid-tightly sealed, and in which a sealing portion, which seals a gap between the inner circumference of the protection body and the outer circumference of the medical elongated body, is provided on a proximal side of the protection body.

According to the protection body and the medical device as described above, the protection layer can prevent external oxygen and moisture from coming into contact with the drug retaining portion. In addition, the absorption portion can absorb oxygen and moisture in a space between the inner circumference of the protection layer and the outer circumference of the drug retaining portion. Accordingly, it can be possible to easily prevent a drug from reacting with oxygen and moisture using a simple structure in which a tubular protection body having the protection layer and the absorption portion covers the outer circumference of the drug retaining portion.

### BRIEF DESCRIPTION OF THE DRAWINGS

FIG. 1 is a view showing a medical device according to a first embodiment.

FIG. 2 is a view showing a state in which a protection sheath is mounted at a distal portion of a balloon catheter.

FIG. 3 is a partially enlarged view showing a distal portion of the medical device.

FIG. 4 is a view showing a medical device according to a second embodiment.

FIG. 5 is a view showing a distal portion of a medical device according to a third embodiment.

FIG. 6(A) is a view of a distal portion of a medical device according to a fourth embodiment which shows a state of the distal portion before a pressing portion performs pressing and FIG. 6(B) is a view showing a state of the distal portion after the pressing portion performs pressing.

FIG. 7 is a view showing a medical device according to Modification Example 1.

FIG. 8 is a view showing a distal portion of a medical device according to Modification Example 2.

FIG. 9 is a view showing a distal portion of a medical device according to Modification Example 3.

FIG. 10 is a view showing a distal portion of a medical device according to Modification Example 4.

### DETAILED DESCRIPTION

### First Embodiment

Hereinafter, a first embodiment of the present disclosure will be described with reference to drawings. Note that the dimensional ratios in the drawings are exaggerated and are different from the actual ratios for the convenience of description. In addition, in the description below, the hand operation side of a medical device 1 according to the first embodiment will be referred to as a “proximal side”, and the side through which the medical device is inserted into a biological lumen will be referred to as a “distal side”.

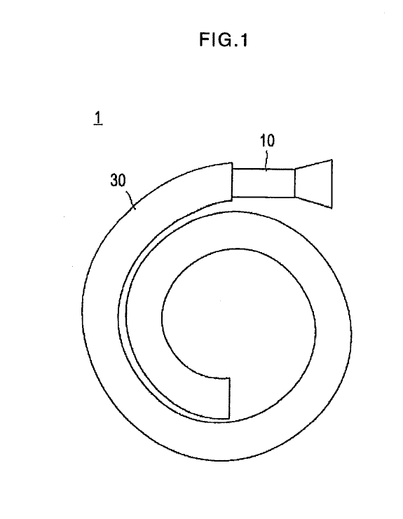

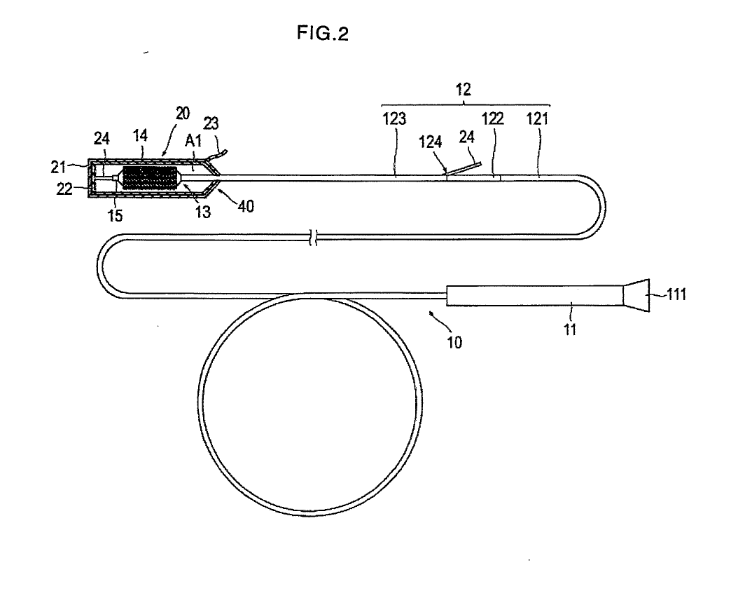

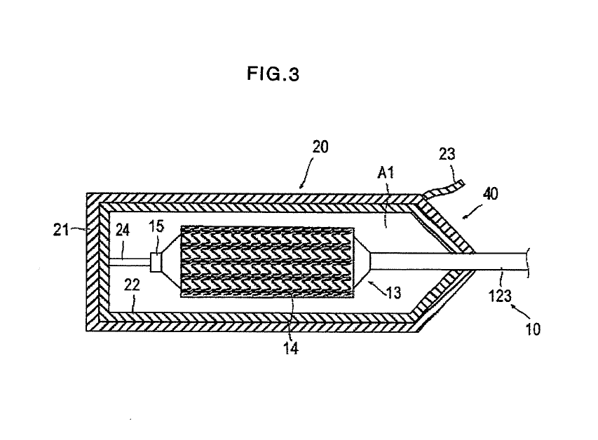

FIG. 1 is a view showing the medical device 1 according to the first embodiment. FIG. 2 is a view showing a state in which a protection sheath 20 is mounted at a distal portion of a balloon catheter 10. FIG. 3 is a partially enlarged view showing a distal portion of the medical device 1. Note that, in FIGS. 2 and 3, the protection sheath 20 is shown by a front cross-sectional view and other configurations are shown by a front view for easy understanding. In addition, a holder tube 30 is not shown in FIG. 3.

The medical device 1 according to the first embodiment has a balloon catheter 10 which serves as a medical elongated body, a protection sheath 20 which serves as a protection body, the holder tube 30, and a sealing portion 40 as shown in FIGS. 1 and 2.

(Holder Tube)

The holder tube 30 is used for protecting the balloon catheter 10, which is accommodated therein as shown in FIG. 1, from contact with the outside, external impacts or the like, and is formed so as to cover the balloon catheter 10. The balloon catheter 10 stored in the holder tube 30 can be taken out by being drawn from a proximal side of the holder tube 30.

(Balloon Catheter)

As shown in FIG. 2, the balloon catheter 10 is a rapid exchange (RX) type balloon catheter having a structure in which a guide wire passes through only a distal portion, and has a hub 11, a shaft portion 12, a balloon 13, a stent 14 which serves as a drug retaining portion, and a tip 15. The shaft′portion 12 includes a proximal shaft 121, an intermediate shaft 122, and a distal shaft 123 from the proximal side.

The hub 11 has an opening 111 to which an indeflator which serves as a pressure application device for supplying a fluid for dilating the balloon 13 can be connected. Examples of the dilation fluid for dilating the balloon 13 include an X-ray contrast agent, a physiological salt solution, and an electrolytic solution, but are not limited thereto.

Examples of the constituent material of the hub 11 include thermoplastic resins such as polycarbonate, polyamide, polysulfone, polyarylate, and a methacrylate-butylene-styrene copolymer.

The proximal shaft 121 has a lumen which communicates with the opening 111 of the hub 11. In addition, the proximal shaft 121 is bonded to the hub 11 in a liquid-tight manner.

The constituent material of the proximal shaft 121 is a metal material, for example, stainless steel, stainless stretchable alloy, Ni—Ti alloy, brass, or aluminum, which has comparatively large rigidity. As necessary, it is also possible to apply a resin material, for example, polyimide, vinyl chloride, or a polycarbonate, which has comparatively large rigidity thereto.

The intermediate shaft 122 has a lumen which communicates with the lumen of the proximal shaft 121. In addition, the intermediate shaft 122 is bonded to the proximal shaft 121 in a liquid-tight manner.

The distal shaft 123 has a lumen which communicates with the lumen of the intermediate shaft 122. In addition, the distal shaft 123 is connected to the intermediate shaft 122 in a liquid-tight manner.

Examples of the constituent materials of the intermediate shaft 122 and the distal shaft 123 include polymer materials such as polyolefins, a cross-linked body of polyolefins, polyvinyl chloride, polyamide, polyamide elastomer, polyester, polyester elastomer, polyurethane, polyurethane elastomer, fluororesin, and polyimide, or a mixture thereof. Examples of the polyolefins include polyethylene, polypropylene, polybutene, an ethylene-propylene copolymer, an ethylene-vinyl acetate copolymer, and an ionomer, or a mixture of two or more kinds thereof.

A guide wire port 124 for introducing a guide wire to a lumen of the distal shaft 123 is provided in a boundary between the intermediate shaft 122 and the distal shaft 123.

The balloon 13 is disposed in a state of being folded (or a state of being contracted), and is dilated by introducing the above-described dilation fluid thereto. The inside of the balloon 13 communicates with the lumen of the distal shaft 123. That is, the inside of the balloon 13 communicates with the opening 111 of the hub 11 through the lumen of the distal shaft 123, the lumen of the intermediate shaft 122, and the lumen of the proximal shaft 121. Accordingly, the dilation fluid which has been made to flow in from the opening 111 of the hub 11 flows into the balloon 13 which is then dilated.

The constituent material of the balloon 13 preferably has flexibility, and examples thereof include polymer materials such as polyolefins, a cross-linked body of polyolefins, polyester, polyester elastomer, polyvinyl chloride, polyurethane, polyurethane elastomer, polyphenylene sulfide, polyamide, polyamide elastomer, and fluororesin, silicone rubber, and latex rubber. Polyester is, for example, polyethylene terephthalate. The constituent material of the balloon 13 is not limited to a form of singly using the above-described polymer material, and it is also possible to apply, for example, a film on which the above-described polymer material is appropriately stacked.

The stent 14 functions as an in-vivo indwelling object which holds the lumen at an appropriate size by being indwelled in a stenosed site as a lesion area by being closely adhered to the inner surface thereof. The stent 14 is expanded in accordance with the dilation of the balloon 13. The stent 14 is a drug eluting stent (DES), in which the outer surface is coated with a drug, and functions as a drug retaining portion in which a drug is retained.

The stent 14 is formed of a material having biocompatibility. Examples of the material having biocompatibility include iron, titanium, aluminum, tin, tantalum or tantalum alloy, platinum or platinum alloy, gold or gold alloy, titanium alloy, nickel-titanium alloy, cobalt-based alloy, cobalt-chromium alloy, stainless steel, zinc-tungsten alloy, and niobium alloy.

A drug with which the outer surface of the stent 14 is coated is a mixture of a biologically/physiologically active substance and a biodegradable polymer. Note that the area to be coated with the drug is not limited to the outer surface of the stent 14, and may be the inner surface of the stent 14, or the outer surface and the inner surface of the stent.

The biologically/physiologically active substance is not particularly limited as long as restenosis or reocclusion of the lumen, which can be caused when the stent 14 according to the present embodiment is indwelled in a stenosed site, is suppressed. The biologically/physiologically active substance can be arbitrarily selected, but it is preferable that the biologically/physiologically active substance is at least one selected from the group consisting of anticancer drugs, immunosuppressors, antibiotics, antirheumatics, antithrombotics, HMG-CoA reductase inhibitors, ACE inhibitors, calcium antagonists, antihyperlipidemics, integrin inhibitors, antiallergics, antioxidants, GPIIbIIIa antagonists, retinoids, flavonoids, carotenoids, lipid-level lowering medicaments, DNA synthesis inhibitors, tyrosine kinase inhibitors, antiplatelets, antiinflammatories, tissue-derived biomaterials, interferons, and NO production promoters, for the reason that it is possible to treat a lesion area by controlling the behavior of cells in tissues of the lesion area.

Preferred examples of the anticancer drug include vincristine, vinblastine, vindesine, irinotecan, pirarubicin, paclitaxel, docetaxel, and methotrexate.

Preferred examples of the immunosuppressors include sirolimus, biolimus A9, tacrolimus, azathioprine, cyclosporine, cyclophosphamide, mycophenolate mofetil, everolimus, ABT-578, AP23573, CCI-779, gusperimus, and mizoribine.

Preferred examples of the antibiotics include mitomycin, adriamycin, doxorubicin, actinomycin, daunorubicin, idarubicin, pirarubicin, aclarubicin, epirubicin, peplomycin, and zinostatin stimalamer.

Preferred examples of the antirheumatics include methotrexate, sodium thiomalate, penicillamine, and lobenzarit.

Preferred examples of the antithrombotics include heparin, aspirin, antithrombotic preparations, ticlopidine, and hirudin.

Preferred examples of the HMG-CoA reductase inhibitors include serivastatin, serivastatin sodium, atorvastatin, rosuvastatin, pitavastatin, fluvastatin, fluvastatin sodium, simvastatin, lovastatin, and pravastatin.

Preferred examples of the ACE inhibitors include quinapril, perindopril erbumine, trandolapril, cilazapril, temocapril, delapril, enalapril maleate, lisinopril, and captopril.

Preferred examples of the calcium antagonists include hifedipine, nilvadipine, diltiazem, benidipine, and nisoldipine.

Preferred examples of the antihyperlipidemics include probucol.

Preferred examples of the integrin inhibitors include AJM300.

Preferred examples of the antiallergics include tranilast.

Preferred examples of the antioxidants include α-tocopherol.

Preferred examples of the GPIIbIIIa antagonists include abciximab.

Preferred examples of the retinoids include all-trans retinoic acids.

Preferred examples of the flavonoids include epigallocatechin, anthocyanine, and proanthocyanidin.

Preferred examples of the carotenoids include β-carotene and lycopene.

Preferred examples of the lipid-level lowering medicaments include eicosapentaenoic acid.

Preferred examples of the DNA synthesis inhibitors include 5-FU.

Preferred examples of the tyrosine kinase inhibitors include genistein, tyrphostin, erbstatin, and staurosporine.

Preferred examples of the antiplatelets include ticlopidine, cilostazol, and clopidogrel.

Preferred examples of the antiinflammatories include steroids such as dexamethasone and prednisolone

Preferred examples of the tissue-derived biomaterials include an epidermal growth factor (EGF), a vascular endothelial growth factor (VEGF), a hepatocyte growth factor (HGF), a platelet derived growth factor (PDGF), and a basic fibrolast growth factor (BFGF).

Preferred examples of the interferons include interferon-γ1a.

Preferred examples of the NO production promoters include L-arginine.

These biologically/physiologically active substances may be used singly or two or more kinds thereof may be used in combination depending on the case.

There is no particular limitation in the biodegradable polymer as long as it is a polymer which gradually degrades when the stent 14 according to the present embodiment is indwelled in a stenosed site and which does not adversely affect a living body. The biodegradable polymer is preferably at least one selected from the group consisting of polyglycolic acids, polylactic acids, polycaprolactone, polyhydroxy butyric acids, cellulose, polyhydroxybutyrate-valerate, and polyorthoesters or a copolymer, a mixture or a compound thereof. This is because these have low reactivity with biological tissues and its degradation in a living body can be controlled.

The tip 15 is disposed at the most distal end of the balloon catheter 10 and is formed to be more flexible than at least the shaft portion 12 for the purpose of protecting the wall surface of blood vessels. The tip 15 may be formed of, for example, polymer materials such as polyolefins (for example, polyethylene, polypropylene, polybutene, an ethylene-propylene copolymer, an ethylene-vinyl acetate copolymer, and an ionomer, or a mixture of two or more kinds thereof), polyvinyl chloride, polyamide, polyamide elastomer, polyurethane, polyurethane elastomer, polyimide, and fluororesin, or a mixture thereof or a multilayered tube of two or more kinds of the above-described polymer materials. Note that the tip 15 may be omitted.

(Protection Sheath)

The stent 14, which is provided at a distal portion of the balloon catheter 10 and retains a drug, is protected by a protection sheath 20. The protection sheath 20 covers and seals the outer circumference and is provided at only the distal portion of the balloon catheter 10.

As shown in FIGS. 2 and 3, the protection sheath 20 has a double-layer structure, and has a protection layer 21 provided on the outer circumference and an absorption layer 22 which serves as an absorption portion provided on the inner circumference. The protection sheath 20 can be formed by, for example, forming the protection layer 21 on the surface of the tubular absorption layer 22 by laminating metal or the like. The protection layer 21 and the absorption layer 22 are provided at only the distal portion of the balloon catheter 10. The protection sheath 20 has a closed shape on the distal side and is liquid-tightly sealed. In addition, the protection sheath 20 has an open shape on the proximal side, is connected to the outer circumference of the distal shaft 123, and constitutes the sealing portion 40 to be described below.

The protection layer 21 is disposed around the outer circumference of the stent 14 along the balloon catheter 10 and prevents external light, oxygen, and moisture from coming into contact with a drug with which the surface of the stent 14 is coated. In addition, the protection layer 21 may have heat shrinkability. The protection layer may be formed by, for example, laminating the surface of a member having heat shrinkability with metal or the like. The material constituting the protection layer 21 is for example, aluminum, but the coating may be performed using silica, alumina, or amorphous carbon. In addition, a film of polyvinylidene (PVDC) can be used as the protection layer 21.

The absorption layer 22 absorbs oxygen and moisture in the space A1 between the inner circumference of the protection layer 21 and the outer circumference of the stent 14. The absorption layer 22 is, for example, a layer in which a deoxidant and a desiccant are implanted in polymer materials such as polyolefins, a cross-linked body of polyolefins, polyvinyl chloride, polyamide, polyamide elastomer, polyester, polyester elastomer, polyurethane, polyurethane elastomer, polyimide, and fluororesin, or a mixture thereof. Examples of the desiccant include silica gel, calcium oxide, and calcium chloride. In addition, examples of the deoxidant include iron powder, sugar, reductone, and catechol. In addition, the absorption layer is not limited to the above-described form, and may be formed such that a deoxidant is implanted in a desiccant formed in a sheet shape. In addition, the absorption layer 22 may have heat shrinkability in a case where the protection layer 21 does not have heat shrinkability.

The inside of the space A1 between the inner circumference of the protection layer 21 and the outer circumference of the stent 14 is preferably purged with nitrogen, but is not always necessarily purged.

As shown in FIG. 3, the protection sheath 20 further has a grip portion 23 and a stylet 24.

The grip portion 23 is provided on the proximal side of the protection layer 21 so as to be connected to the protection layer 21, and is gripped when separating the protection layer 21 as will be described below. By moving the grip portion 23 after gripping the grip portion 23 to the distal side, the protection layer 21 and the absorption layer 22 are separated. The grip portion 23 may be provided so as to be connected to the absorption layer 22. Note that the grip portion is preferably connected and provided to the absorption layer 22 which is a layer on the inside of the protection sheath 20 from the viewpoint of separating the protection sheath 20.

As shown in FIG. 2, the distal side of the stylet 24 is connected to the distal side of the inner circumference of the absorption layer 22, extends to the tip 15 and inside of the balloon 13 and the inside of the lumen of the distal shaft 123, and is derived to the outside of the medical device 1 through the guide wire port 124. The stylet 24 extends substantially on the center axis of the protection sheath 20. Accordingly, it is possible to dispose the balloon 13 on the center axis of the protection sheath 20. In addition, it is possible to insert the stylet 24 into the inside of the balloon 13 and the lumen of the distal shaft 123, and therefore, it is possible to freely insert the protection sheath 20 to or extract the protection sheath from the distal portion of the balloon catheter 10. In addition, it is preferable that the outer diameter of the stylet 24 and the inner diameter of the guide wire port 124 are substantially equal to each other in order to prevent external oxygen and moisture from entering the space A1 through the guide wire port 124 and the lumen of the distal shaft 123. As a specific example, the outer diameter of the stylet 24 is, for example, 0.4 mm and the inner diameter of the guide wire port 124 is, for example, 0.405 mm.

The constituent material of the stylet 24 preferably has flexibility, and examples thereof include polymer materials such as polyolefins, a cross-linked body of polyolefins, polyvinyl chloride, polyamide, polyamide elastomer, polyester, polyester elastomer, polyurethane, polyurethane elastomer, polyimide, and fluororesin, or a mixture thereof.

(Sealing Portion)

As shown in FIGS. 2 and 3, the sealing portion 40 seals the gap between the inner circumference of the end portion on the proximal side of the protection sheath 20 and the outer circumference of the balloon catheter 10. The sealing portion 40 is formed by contracting the protection layer 21 by adding heat to the proximal side of the protection sheath 20. It is possible to set the space A1 to be in a closed system using the sealing portion 40 by sealing the gap between the inner circumference of the end portion on the proximal side of the protection sheath 20 and the outer circumference of the balloon catheter 10.

(Manufacturing Method and Indwelling Method)

Next, a method for manufacturing the medical device 1 and a method for indwelling the stent 14 will be described.

In manufacturing the medical device 1, the balloon catheter 10 is stored in the holder tube 30 in a state in which the stent 14 coated with a drug is mounted on the balloon 13 and the protection sheath 20 is mounted at the distal portion to thereby form the medical device 1. Then, the medical device 1 is sealed in a package and is sterilized using an electron beam or the like. In a case where the package is a material of blocking an electron beam, sterilizing means using other radioactive rays such as a γ-ray may be used. The package is, for example, a polyethylene bag.

In order to use the medical device 1 in a procedure involving indwell the stent 14, an operator will first take the medical device 1 out of the package. Next, the balloon catheter 10 is taken out of the holder tube 30, the protection layer 21 and the absorption layer 22 are separated by moving the grip portion 23 to the distal side while gripping the grip portion 23, the protection sheath 20 is slid to the distal side and is removed from the balloon catheter 10, and the stylet 24 is removed from the distal portion of the medical device 1.

A guide wire is then inserted into the lumen of a patient, and the distal portion of the medical device 1 is inserted into the lumen along the guide wire which has been inserted thereinto, and is positioned at a stenosed site as a target site.

Next, a dilation fluid is introduced from the opening 111 of the hub 11 into the balloon 13 through the lumen of the proximal shaft 121, the lumen of the intermediate shaft 122, and the lumen of the distal shaft 123 to dilate the balloon 13. The dilation of the balloon causes expansion and plastic deformation of the stent 14 which is closely adhered to the stenosed site.

Thereafter, the balloon 13 is contracted by reducing the pressure after taking the dilation fluid out of the inside of the balloon 13. As a result, the connection between the stent 14 and the balloon 13 is released, and therefore, it is possible to separate the stent 14 from the balloon 13. Accordingly, the stent 14 is indwelled in the stenosed site. Finally, the medical device 1 from which the stent 14 is separated is retracted and is removed from the lumen.

(Effect)

As described above, the protection sheath 20 protects the outer circumference of the stent 14 which is provided at the distal portion of the balloon catheter 10 and in which a drug is retained, by covering and sealing the outer circumference. The protection sheath 20 has the protection layer 21 which is disposed around the outer circumference of the stent 14 along the balloon catheter 10 and can prevent external oxygen and moisture from coming into contact with a drug with which the surface of the stent 14 is coated, and the absorption layer 22 which can absorb oxygen and moisture in the space A1 between the inner circumference of the protection layer 21 and the outer circumference of the stent 14. Accordingly, it is possible to easily prevent a drug from reacting with oxygen and moisture using a simple configuration in which the tubular protection sheath 20 having the protection layer 21 and the absorption layer 22 covers the outer circumference of the stent 14.

In addition, the protection layer 21 and the absorption layer 22 are provided on only the distal side of the balloon catheter 10. For this reason, the protection layer 21 and the absorption layer 22 are provided in only the necessary minimum range. Accordingly, it is possible to provide the protection sheath 20 at low cost. Furthermore, even if an impact is received in transit or the like, the range in which the protection sheath 20 is provided is narrow. Thus, it is difficult to accidentally generate a pinhole on the protection layer 21 of the protection sheath 20, and therefore, it is possible to reduce a possibility that a drug may react with oxygen or moisture. In addition, since it is difficult to accidentally generate a pinhole on the protection layer 21 of the protection sheath 20, it is also possible to reduce irradiation of a drug with light.

In addition, the absorption layer 22 is in a layer shape and is provided so as to come into contact with the inner peripheral surface of the protection layer 21. According to this configuration, the absorption layer 22 which has been formed in a layer shape may come into contact with the inner peripheral surface of the protection layer 21, and manufacturing of the protection sheath 20 is facilitated.

In addition, the protection sheath further has the grip portion 23 which is provided so as to be connected to the protection layer 21 or the absorption layer 22 and is gripped when separating the protection layer 21. Accordingly, an operation to remove the protection sheath 20 at the time of using the medical device 1 is facilitated.

In addition, the medical device 1 according to the present embodiment has the above-described protection sheath 20, and the balloon catheter 10 including the stent 14, in which a drug is retained, at the distal portion. The inner circumference of the protection sheath 20 on the distal side is liquid-tightly sealed, and a sealing portion 40, which seals a gap between the inner circumference of the protection sheath 20 and the outer circumference of the balloon catheter 10, is provided on the proximal side of the protection sheath 20. According to this configuration, since the balloon catheter has the above-described protection sheath 20, it is possible to easily prevent a drug for preventing restenosis of a lesion area from reacting with oxygen and moisture.

In addition, the protection sheath 20 can have heat shrinkability, in which case the sealing portion 40 is formed by contracting the protection sheath 20 by adding heat thereto. For this reason, it is possible to reliably seal the gap between the inner circumference of the end portion on the proximal side of the protection sheath 20 and the outer circumference of the balloon catheter 10 through a simple method. Note that, in a case where the protection sheath 20 does not have heat shrinkability, a heat-shrinkable tube may be covered from the outer surface of the protection sheath 20 on the proximal side over the outer surface of the balloon catheter, and the sealing portion may be formed by contracting the heat-shrinkable tube by adding heat thereto.

### Second Embodiment

Next, a second embodiment will be described. The portions common to the first embodiment will not be described, and only features which differ in the second embodiment will be described. In a medical device 2 according to the second embodiment, a holder tube 130 functions as a protection body. Hereinafter, the medical device 2 according to the second embodiment will be described in detail.

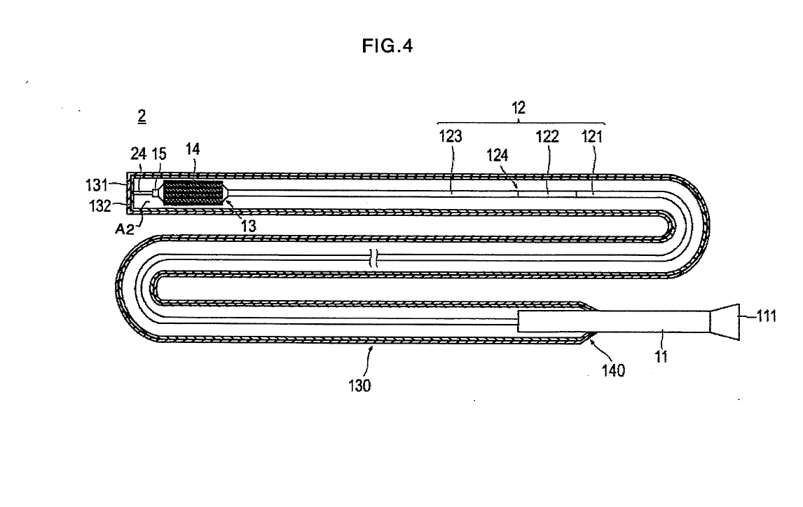

FIG. 4 is a view showing the medical device 2 according to the second embodiment. Note that, in FIG. 4, the holder tube 130 is shown by a front cross-sectional view and other configurations are shown by a front view for easy understanding.

The medical device 2 according to the second embodiment has the balloon catheter 10, the holder tube 130 as a protection body, and a sealing portion 140 as shown in FIG. 4.

The holder tube 130 extends to the proximal side of the balloon catheter 10. The holder tube 130 has a protection layer 131 which is disposed around the outer circumference of the stent 14 along the balloon catheter 10 and can prevent external oxygen and moisture from coming into contact with a drug with which the surface of the stent 14 is coated, and an absorption layer 132 which can absorb oxygen and moisture in a space A2 between the inner circumference of the protection layer 131 and the outer circumference of the stent 14. The protection layer 131 and the absorption layer 132 extend further on a proximal side than the guide wire port 124.

The sealing portion 140 seals the gap between the inner circumference of an end portion on the proximal side of the holder tube 130 and the outer circumference of the balloon catheter 10. The sealing portion 140 is formed by contracting the protection layer 131 by adding heat to a proximal portion of the holder tube 130.

Note that, in the second embodiment, the holder tube 130 is sealed by the guide wire port 124 on the proximal side. Accordingly, oxygen and moisture do not enter from the outside through the guide wire port 124, and therefore, the stylet 24 may not extend to the guide wire port 124 from the distal portion of the balloon catheter 10. Similarly, since oxygen and moisture do not enter from the outside through the guide wire port 124, the outer diameter of the stylet 24 may be smaller than the inner diameter of the guide wire port 124. With such a configuration, it is possible to reduce the risk in which lubricating coatings or the like provided on the inner surface of the guide wire port 124 are peeled off, by reducing the contact of the stylet 24 with the inner surface of the guide wire port 124. It is also possible that the stylet 24 is not provided at all in this embodiment. However, it is preferable to provide the stylet 24 in order to help protect the balloon shape and the lumen of the guide wire port 124. In the case that the stylet 24 is provided, it is removed from the distal portion of balloon catheter 10 after removal of the holder tube 130 from the balloon catheter 10.

As described above, in the medical device 2 according to the second embodiment, the protection layer 131 and the absorption layer 132 extend to the proximal side of the balloon catheter 10. According to this configuration, the holder tube 130 can perform a function as a protection body. Accordingly, it is possible to reduce the number of parts of the medical device 2, and therefore, it is possible to provide the medical device 2 at low cost.

### Third Embodiment

Next, a third embodiment will be described. The portions common to the first embodiment will not be described, and only features which differ in the third embodiment will be described. A medical device 3 of the third embodiment is different from the medical device 1 according to the first embodiment in that an absorption member 222 which serves as an absorption portion is provided on the inner circumference of the protection layer 221. Hereinafter, the medical device 3 according to the third embodiment will be described in detail.

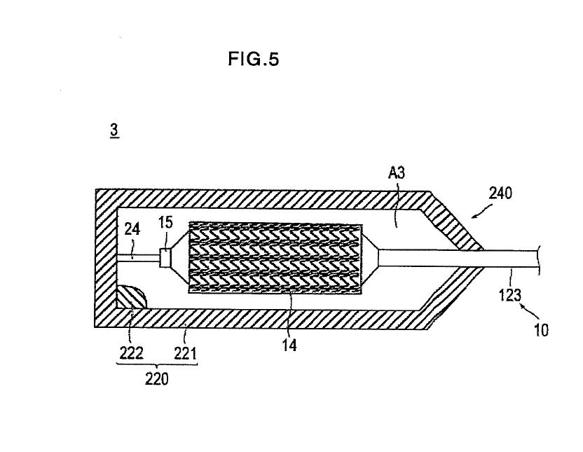

FIG. 5 is a view showing a distal portion of the medical device 3 according to the third embodiment. Note that, in FIG. 5, the protection sheath 220 is shown by a front cross-sectional view and other configurations are shown by a front view for easy understanding. In addition, the holder tube 30 is not shown in FIG. 5.

The medical device 3 according to the third embodiment has the balloon catheter 10, the protection sheath 220 which serves as a protection body, and a sealing portion 240 as shown in FIG. 5.

The protection sheath 220 has a protection layer 221 which is provided around the outer circumference of the stent 14 along the balloon catheter 10 and can prevent external oxygen and moisture from coming into contact with a drug with which the surface of the stent 14 is coated. In addition, the absorption member 222 which can absorb oxygen and moisture in a space A3 between the inner circumference of the protection layer 221 and the outer circumference of the stent 14 is provided on the distal side of the inner circumference of the protection layer 221. The site on which the absorption member 222 is provided is not limited as long as the site is on the inner circumference side of the protection layer 221. The protection layer 221 has heat shrinkability. The absorption member 222 is, for example, silica gel. The sealing portion 240 is formed by contracting the protection layer 221 by adding heat thereto similarly to the sealing portion 40 according to the first embodiment.

As described above, the absorption member 222 is provided on the inner circumference side of the protection layer 221 in the medical device 3 according to the third embodiment. Accordingly, it is possible to adjust the amount of the absorption member 222 as necessary, and therefore, it is possible to more reliably absorb oxygen and moisture in the space A3 between the inner circumference of the protection layer 221 and the outer circumference of the stent 14.

### Fourth Embodiment

Next, a fourth embodiment will be described. The portions common to the first embodiment will not be described, and only features which differ in the fourth embodiment will be described. A medical device 4 according to the fourth embodiment is different from the medical device 1 according to the first embodiment in terms of the configuration of a sealing portion 340. Hereinafter, the medical device 4 according to the fourth embodiment will be described in detail.

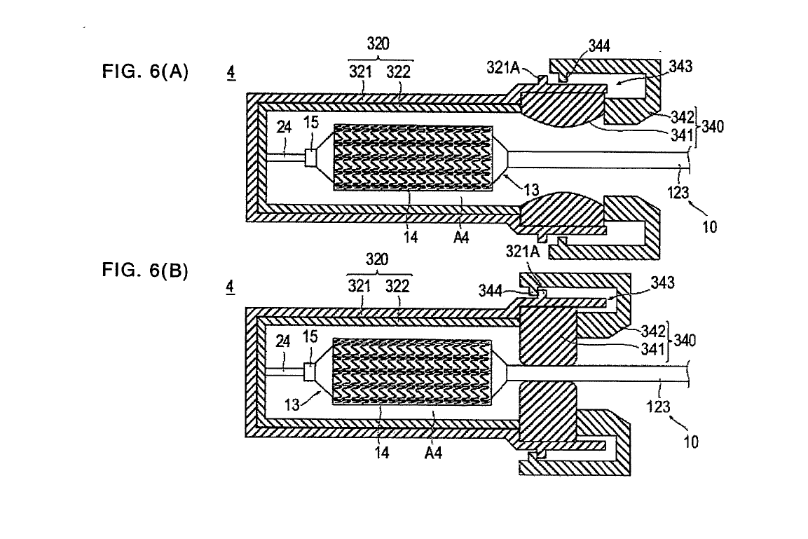

FIG. 6(A) is a view of a distal portion of a medical device according to a fourth embodiment which shows a state of the distal portion before a pressing portion performs pressing and FIG. 6(B) is a view showing a state of the distal portion after the pressing portion performs pressing. Note that, in FIG. 6, a protection sheath 320 and the sealing portion 340 are shown by a front cross-sectional view and other configurations are shown by a front view for easy understanding. In addition, the holder tube 30 is not shown in FIGS. 6(A) and 6(B).

The medical device 4 according to the fourth embodiment has the balloon catheter 10, the protection sheath 320, and the sealing portion 340 as shown in FIGS. 6(A) and 6(B).

The protection sheath 320 has the protection layer 321 which is disposed around the outer circumference of the stent 14 along the balloon catheter 10 and can prevent external oxygen and moisture from coming into contact with a drug with which the surface of the stent 14 is coated, and an absorption layer 322 which can absorb oxygen and moisture in a space A4 between the inner circumference of the protection layer 321 and the outer circumference of the stent 14.

The protection layer 321 is provided so as to extend further on a proximal side than the absorption layer 322. The protection layer 321 has a projection portion 321A on the outer circumference on the proximal side.

The sealing portion 340 has an elastic body 341 provided on the inner surface of the protection layer 321 on the proximal side, and a pressing portion 342 which presses the elastic body 341. The elastic body 341 is, for example, rubber, but the present invention is not limited thereto.

The pressing portion 342 has a concave portion 343 into which the proximal side of the protection layer 321 is inserted. In addition, a projection portion 344 is provided on the outer circumference side of the concave portion 343.

In the above-described configuration, the pressing portion 342 presses the elastic body 341 by moving the pressing portion 342 in a left direction in FIG. 6(A) such that the proximal side of the protection layer 321 enters the concave portion 343 of the pressing portion 342, and the projection portion 344 of the pressing portion 342 is engaged with the projection portion 321A of the protection layer 321. As a result, the elastic body 341 abuts on the outer circumference of the distal shaft 123 as shown in FIG. 6(B), and the gap between the inner circumference in the end portion on the proximal side of the protection sheath 320 and the outer circumference of the balloon catheter 10 is sealed.

In the present embodiment, when removing the protection sheath 320 from the balloon catheter 10 at the time of using the medical device 4, first, the pressing portion 342 is detached by being slid to the proximal side, and then, the removal is performed by sliding the protection sheath 320 and the elastic body 341 to the distal side.

As described above, in the medical device 4 according to the fourth embodiment, the sealing portion 340 has the elastic body 341 and the pressing portion 342 which presses the elastic body 341. The gap between the inner circumference of the end portion on the proximal side of the protection sheath 320 and the outer circumference of the balloon catheter 10 is sealed by the pressing portion 342 pressing the elastic body 341. For this reason, it is possible to more reliably seal the gap between the inner circumference in the end portion on the proximal side of the protection sheath 320 and the outer circumference of the balloon catheter 10.

### Modification Example 1

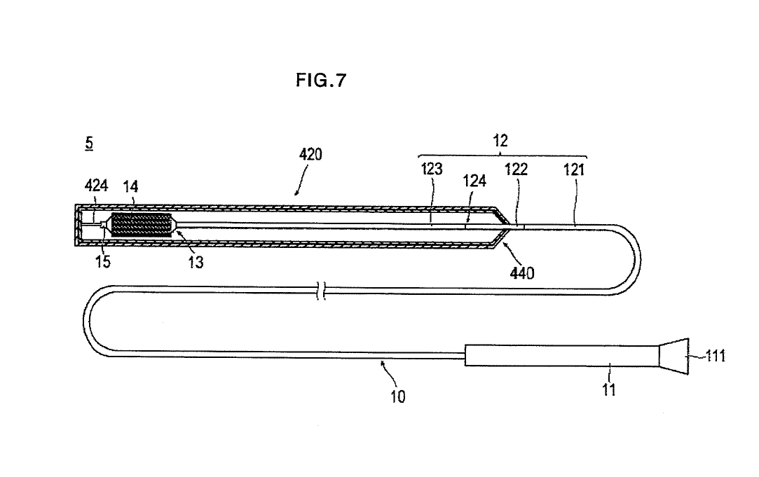

FIG. 7 is a view showing a medical device 5 according to Modification Example 1. In FIG. 7, the protection sheath 420 is shown by a front cross-sectional view and other configurations are shown by a front view for easy understanding. In addition, the holder tube 30 is not shown in FIG. 7. In the above-described first embodiment, the protection sheath 20 is provided on only the distal side of the balloon catheter 10. However, the protection sheath 420 of the medical device 5 may extend further on a proximal side than the guide wire port 124 as shown in FIG. 7. At this time, a sealing portion 440 is connected to the outer circumference of the intermediate shaft 122. According to this configuration, it is possible to prevent oxygen and moisture from entering the protection sheath 420 from the outside through the guide wire port 124, and therefore, it is possible to more reliably prevent a drug from reacting with oxygen and moisture. In addition, at this time, the stylet 424 does not need to extend until the stylet blocks the guide wire port 124.

### Modification Example 2

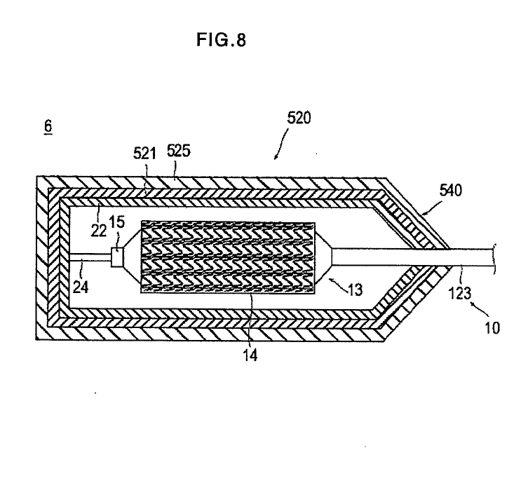

FIG. 8 is a view showing a distal portion of a medical device 6 according to Modification Example 2. In FIG. 8, a protection sheath 520 is shown by a front cross-sectional view and other configurations are shown by a front view for easy understanding. In addition, the holder tube 30 is not shown in FIG. 8. In the above-described first embodiment, the protection sheath 20 has heat shrinkability, and the sealing portion 40 is formed by adding heat to the protection sheath 20. However, as shown in FIG. 8, a sealing portion 540 may be formed by further providing a heat shrinkable tube 525 on the outer circumference of a protection layer 521 and contracting this heat shrinkable tube 525 by adding heat thereto. At this time, the protection layer 521 and the absorption layer 22 may or may not have heat shrinkability. Note that the heat shrinkable tube 525 may be disposed from the proximal side of the protection layer 521 over the distal shaft 123 without having a configuration of covering the entirety of the protection layer 521.

### Modification Example 3

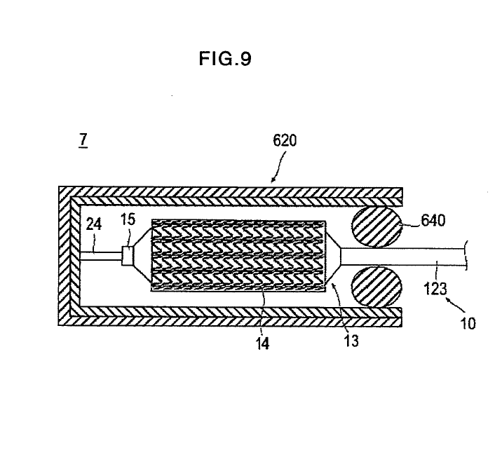

FIG. 9 is a view showing a distal portion of a medical device 7 according to Modification Example 3. In FIG. 9, a protection sheath 620 is shown by a front cross-sectional view and other configurations are shown by a front view for easy understanding. In addition, the holder tube 30 is not shown in FIG. 9. In the above-described first embodiment, the sealing portion 40 is formed by adding heat to the protection sheath 20. However, a sealing portion 640 of the medical device 7 may be water-soluble gel molecules which are provided in the gap between the inner circumference of the end portion on the proximal side of the protection sheath 620 and the outer circumference of the balloon catheter 10 as shown in FIG. 9. Examples of the water-soluble gel molecules include polyacrylic acids, polyethylene oxide, and polyvinyl alcohol. In Modification Example 3, when removing the protection sheath 620 from the balloon catheter 10 at the time of using the medical device 7, first, the sealing portion 640 is dissolved by immersing the distal portion of the medical device 7 in physiological salt solution, and then, the removal is performed by sliding the protection sheath 620 to the distal side. Therefore, according to this configuration, it is possible to remove the protection sheath 620 through a simple method.

### Modification Example 4

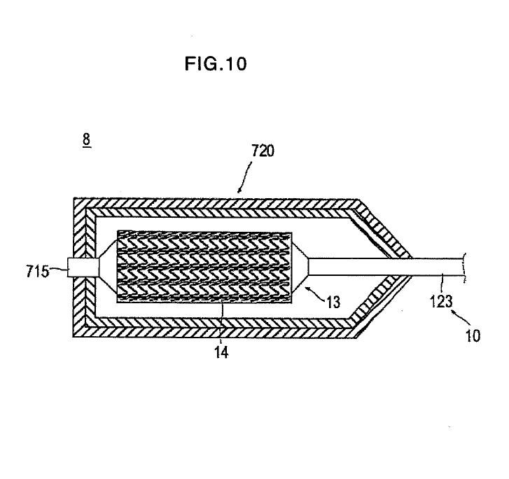

FIG. 10 is a view showing a distal portion of a medical device 8 according to Modification Example 4. In FIG. 10, a protection sheath 720 is shown by a front cross-sectional view and other configurations are shown by a front view for easy understanding. In addition, the holder tube 30 and the stylet 24 are not shown in FIG. 10. In the above-described first embodiment, the protection sheath 20 is provided so as to cover the entirety of the tip 15. However, the protection sheath 720 may be connected to the outer circumference of a tip 715 as shown in FIG. 10. According to this configuration, there is no case where oxygen and moisture come into contact with the stent 14 even if oxygen and moisture enter the lumen of the distal shaft 123 from the outside through the guide wire port 124. Accordingly, it is possible to make the outer diameter of the stylet 24 be smaller than the inner diameter of the guide wire port 124. For this reason, it is possible to reduce the risk in which lubricating coatings or the like provided on the inner peripheral surface of the guide wire port 124 are peeled off, by reducing the contact of the stylet 24 with the inner peripheral surface of the guide wire port 124.

### Modification Example 5

In the above-described fourth embodiment, the elastic body 341 is pressed by moving the pressing portion 342 in the left direction in FIG. 6. However, the outer circumference of the protection layer on the proximal side and the outer circumference of the concave portion of the pressing portion may be formed in a spiral shape, and the elastic body may be pressed by rotating the pressing portion.

### Modification Example 6

In the above-described first embodiment, the protection sheath 20 has a double-layer structure of the protection layer 21 and the absorption layer 22. However, the present invention is not limited thereto, and the protection sheath may be constituted of three or more layers by combining the protection layer 21 and the absorption layer 22, or a protection layer may have appropriate absorbing properties and thereby also serve as an absorption portion.

### Modification Example 7

In the above-described first to fourth embodiments and modification examples 1 to 6, the protection sheaths and the protection bodies are applied to a drug eluting stent (DES). However, the present invention is not limited thereto, and the protection sheaths or the protection bodies can also be applied to a drug eluting balloon (DEB) in which the outer circumference of the balloon is coated with a drug. Note that, in the case of the drug eluting balloon (DEB), the balloon corresponds to the drug retaining portion.

### Modification Example 8

In the above-described first embodiment, the balloon catheter 10 is a rapid exchange type balloon catheter. However, the present invention is not limited thereto and can also be applied to an over-the-wire (OTW) type balloon catheter. In this case, the balloon catheter has a structure in which a guide wire passes from a distal end to the hand, and therefore, exchange of the guide wire or the operability is improved. In addition, the balloon catheter is not limited to a form in which the balloon catheter is applied to a stenosed site generated in the coronary artery in the heart, and can also be applied to a stenosed site generated in other blood vessels, the bile duct, the trachea, the esophagus, the urethra, or the like.

The detailed description above describes a protection body and medical device. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A protection body protecting an outer circumference of a drug retaining portion, which is provided at a distal portion of a medical elongated body and in which a drug is retained, by covering and sealing the outer circumference, comprising:
a protection layer which is disposed around the outer circumference of the drug retaining portion along the medical elongated body and is configured to prevent external oxygen and moisture from coming into contact with the drug retaining portion; and
an absorption portion which is configured to absorb oxygen and moisture in a space between an inner circumference of the protection layer and the outer circumference of the drug retaining portion.

2. The protection body according to claim 1,
wherein the protection layer and the absorption portion are provided only at the distal portion of the medical elongated body.

3. The protection body according to claim 1,
wherein the medical elongated body is a rapid exchange type catheter having a guide wire port, and
wherein the protection layer and the absorption portion extend further on a proximal side than the guide wire port.

4. The protection body according to claim 1,
wherein the absorption portion has a layer shape and is provided so as to come into contact with an inner peripheral surface of the protection layer.

5. The protection body according to claim 1,
wherein the absorption portion is provided on an entire inner circumference of the protection layer.

6. The protection body according to claim 1, further comprising:
a grip portion which is provided so as to be connected to the protection layer or the absorption portion and is gripped when separating the protection layer.

7. A medical device comprising:
a medical elongated body including a drug retaining portion, in which a drug is retained, at a distal portion; and
a protection body protecting an outer circumference of the drug retaining portion by covering and sealing the outer circumference, said protection body including a protection layer which is disposed around the outer circumference and is configured to prevent external oxygen and moisture from coming into contact with the drug retaining portion, and an absorption portion which is configured to absorb oxygen and moisture in a space between an inner circumference of the protection layer and the outer circumference of the drug retaining portion,
wherein the inner circumference of the protection body on a distal side is liquid-tightly sealed, and
wherein a sealing portion, which seals a gap between the inner circumference of the protection body and the outer circumference of the medical elongated body, is provided on a proximal side of the protection body.

8. The medical device according to claim 7,
wherein the protection body has thermal contraction properties, and
wherein the sealing portion is formed by making the protection body contract by adding heat thereto.

9. The medical device according to claim 7,
wherein the sealing portion has an elastic body and a pressing portion which presses the elastic body, and
wherein the gap is sealed by the pressing portion pressing the elastic body.

10. The medical device according to claim 7,
wherein the sealing portion is formed of water-soluble gel molecules disposed in the gap.

11. A method of manufacturing a packaged medical device, comprising:
mounting a protection body to a drug-retaining portion of a medical elongated body to form a medical device, said protection body comprising a protection layer which is disposed around an outer circumference of the drug-retaining portion and is configured to prevent external oxygen and moisture from coming into contact with the drug retaining portion, and an absorption portion which is configured to absorb oxygen and moisture in a space between an inner circumference of the protection layer and the outer circumference of the drug retaining portion;
sealing the medical device in a package to form a packaged medical device; and
sterilizing the packaged medical device.

12. The method according to claim 11, wherein the sterilizing is performed using an electron beam.

13. The method according to claim 11, wherein the sterilizing is performed using a γ-ray.

14. The method according to claim 11, further comprising sealing a gap between an inner circumference of the protection body and an outer circumference of the medical elongated body by adding heat to the protection body to cause the protection body to contract.

15. The method according to claim 11, further comprising purging the space between an inner circumference of the protection layer and the outer circumference of the drug retaining portion with nitrogen.
